(19) United States
(12) Patent Application Publication
(10) Pub. No.: US 2004/0223931 A1
(43) Pub. Date: Nov. 11, 2004

(54) Exfoliated phyllosilicate-based cosmetic compositions

(75) Inventors: Jean Mondet, Aulnay Sous Bois (FR); Philippe Ilekti, Paris (FR)

Correspondence Address: OLIFF & BERRIDGE, PLC, P.O. BOX 19928, ALEXANDRIA, VA, 22320, US

(73) Assignee: L'OREAL, Paris (FR)

(21) Appl. No.: 10/838,328

(22) Filed: May 5, 2004

Related U.S. Application Data
(60) Provisional application No. 60/472,750, filed on May 23, 2003.

(30) Foreign Application Priority Data
May 5, 2003 (FR) 03 05446

(51) Int. Cl. A61K 7/025

(52) U.S. Cl. 424/64

(57) ABSTRACT

A cosmetic composition for caring for and/or making up the skin, lips and/or superficial body growths which is notably translucent or transparent and/or which is capable of giving a translucent or transparent coat includes at least one liquid fatty phase or at lest one one water-immiscible organic phase, with the phase being structured by at least an effective amount of exfoliated phyllosilicate derived from at least one intercalated phyllosilicate.

### CROSS-REFERENCE TO RELATED APPLICATIONS

This application claims the benefit of French Application No. 03 05446 filed on May 5, 2003 and U.S. Provisional Application No. 60/472,750 filed on May 23, 2003, the entire disclosures of which are incorporated by reference herein.

### BACKGROUND OF THE INVENTION

The present invention relates to thickened, indeed even gelled, cosmetic compositions referred to as translucent or transparent and which can be colored, for caring for and/or making up the skin, lips and superficial body growths, the coats of which may be notably also translucent or transparent.

More particularly, the compositions considered according to the invention can constitute products for making up the skin, lips and/or superficial body growths, especially the nails, having in particular nontherapeutic care and/or treatment properties. The cosmetic products more particularly concerned are, for example, lipsticks, mascaras, eyeliners, foundations, powders, blushes, eyeshadows, nail varnish, products for nail care and products for making up the body.

Generally, cosmetic compositions, whether intended for making up the skin, such as foundations, lipsticks, eyeshadows or blushes, or the superficial body growths, such as mascaras, or nail varnish, are intended above all to provide the make-up substrate with an aesthetic effect. The effect most often desired is a colored effect of glossiness and/or mattness, more particularly in the case of foundations, the latter being intended in particular to effectively mask skin imperfections, such as microreliefs, wrinkles, fine lines, pores or color variations.

Conventionally, the above mentioned make-up compositions are applied to the surface to be made up in the form of a coat intended to provide the expected effects of coloration and/or of glossiness and/or of mattness.

In point of fact, the user is increasingly looking to reconcile the above effects with a “natural” effect. The make-up applied, in particular to the skin, must provide the desired aesthetic effect, namely a colored, glossy and/or matt effect, while conferring the illusion of a natural appearance on the make-up substrate. In other words, it would be desirable for the make-up coat to be invisible or for the cosmetic make-up and/or care composition to be, after application as a fine layer, completely transparent or else sufficiently translucent to retain a natural effect of the skin.

Colored make-up compositions described as transparent or translucent, the coat of which on the make-up substrate is transparent or translucent, have already been provided. These compositions generally possess a liquid fatty phase or a water-immiscible organic phase structured by a texturizing agent other than a conventional organic or inorganic filler or wax, which exhibits specifically the disadvantage of rendering the cosmetic composition opaque. In the specific case of fillers, this undesirable effect is related in particular to their size, generally on the scale of a micron, and/or to their natural mattness.

The structuring agents provided are generally organic in nature, namely polymers (EP 1 068 856), gellifying polyamides (WO 00/247,627), esters or amides of N-acylamino acids (WO 01/97758) or silicone derivatives (WO 97/36573, WO 02/17870, WO 02/17871, U.S. Pat. No. 6,051,216). However, the use of these compounds is not entirely satisfactory. In particular, some of them confer a sticky nature, of course undesirable, on the corresponding cosmetic compositions.

### SUMMARY OF THE INVENTION

The inventors have discovered, surprisingly, that exfoliated phyllosilicates, notably deriving from at least one phyllosilicate, preferably combined with cosmetic components make it possible to obtain particularly useful properties. Among such properties achieved in various embodiments of the invention may be mentioned thickened, or even gelled, compositions, the coat or film of which on the make-up substrate can be translucent or transparent, while maintaining good texturizing properties, good properties of hold, of glossiness, of mattness and absence of migration. One or more of these and other desirable properties can be obtained through use of such exfoliated phyllosilicates.

Exfoliated phyllosilicates constitute a particularly advantageous additive in the field of cosmetics for providing coats of cosmetic compositions that can be translucent or transparent and, in addition, advantageously possess good properties of hold over time and are devoid of any sticky nature.

Phyllosilicates, more commonly known as clays, can refer to layered silicates.

The term “intercalated phyllosilicates” can refer to phyllosilicates that have been treated with organic or inorganic compounds for the purpose of introducing molecules of these compounds into the interlayer spaces of the phyllosilicates, on the one hand to increase the distance between the sheets and, on the other hand, to give them an organophilic nature. Generally, a sufficient amount of molecules may be adsorbed between two adjacent sheets of phyllosilicates so as to increase their interlayer space, for example, to a size of at least 5 angstroms, in particular of approximately 10 angstroms, and thus to promote the resulting exfoliation of the intercalated material in the form of separate sheets. This exfoliation, or alternatively delamination, can be carried out under shearing in the presence of an organic solvent or in a polymeric matrix to obtain “exfoliated phyllosilicates”.

At the current time, two exemplary intercalation techniques are preferred.

The first technique, disclosed, for example, in WO 93/04118, includes exchanging hydrophilic inorganic cations, originally present between the phyllosilicate sheets, with organic “onium” cations, generally quaternary alkylammoniums. The phyllosilicates thus intercalated are used in their exfoliated form in polymeric matrices for the purpose of reinforcing their mechanical properties. Quaternary ammonium cations are well known for their use in converting very hydrophilic phyllosilicates, such as calcium or sodium montmorillonites, into organophilic phyllosilicates.

The second technique is targeted at modifying the nature of the ligands to which the cations of the interlayer spaces are coordinated. In the natural state, the cations of the interlayer spaces are coordinated to water molecules. This second method is targeted at substituting, for these water molecules, specific organic hydrocarbonaceous molecules comprising at least one polar group. U.S. Pat. No. 5,721,306 discloses, as intercalating agents, hydrocarbonaceous molecules having at least one polar group of hydroxyl, carbonyl, carboxyl, amine, amide, ether, ester and indeed even also sulfate, sulfonate, sulfinate, sulfamate, phosphate, phosphonate or phosphinate type or one aromatic group, capable of interacting with metal cations bonded to the surfaces of the phyllosilicate sheets. They are in particular alkylpyrrolidone, polyvinylpyrrolidone, polyvinyl alcohol polyoxyalkylenes, polyamide or polyimide derivatives. The phyllosilicates thus intercalated are subsequently exfoliated. They are provided in U.S. Pat. No. 5,721,306 as thickening agents for preparing formulations of viscous thixotropic gel type, indeed even solid gels, and in particular as vehicles for a wide variety of active materials. U.S. Pat. No. 6,500,411 discloses the use of phyllosilicates intercalated and exfoliated using natural polyphenol derivatives, like lignin, for example, in aqueous cosmetic compositions, in particular of antisun formulation type.

In various exemplary embodiments, the present invention is directed to a cosmetic composition for caring for and/or making up the skin, lips and/or superficial body growths, comprising a physiologically acceptable medium containing at least one liquid fatty phase and/or one water immiscible organic phase, the phase being structured by at least an effective amount of exfoliated phyllosilicates deriving from at least one intercalated phyllosilicate.

As used herein, the term “derived” in the expression “exfoliated phyllosilicates derived from at least one intercalated phyllosilicate” can refer to the fact that phyllosilicates in a sheet-form can be obtained from intercalated phyllosilicates following the application of an efficient shear stress allowing at least partial, and perhaps total, exfoliation. The nature of shear stress suitable for application is further detailed bellow.

According to a particular embodiment of the invention, the physiologically acceptable medium comprises at least one liquid fatty phase.

In various exemplary embodiments, the composition is an emulsion.

In various exemplary embodiments, emulsions containing exfoliated phyllosilicates in accordance with the invention may comprise a smaller amount of emulsifying and co-emulsifying agents, indeed even may be totally free from those agents.

The inventors have thus discovered that exemplary phyllosilicates in accordance with the invention can be advantageously used as stabilizing agent to control the stability of cosmetic emulsions, which ordinarily require an efficient amount of surfactants or emulsifying and co-emulsifying agents. Phyllosilicates allow significant or complete reductions of the need for surfactants or emulsifying and co-emulsifying agents usually required, with an obvious advantage with respect to innocuousness.

In various exemplary embodiments, compositions according to the invention may be advantageously transparent or translucent and/or capable of giving a transparent or translucent coat.

As used herein, a property of “bulk” transparency or translucency can refer to a layer of a composition with a given thickness that allows a portion of visible light to pass. If this portion of visible light is scattered, the composition can be defined as a bulk translucent composition and if, on the other hand, it is not scattered, then the composition can be defined as a bulk transparent composition.

In various exemplary embodiments, at least one intercalated phyllosilicate in accordance with the invention can be used as an agent for texturizing a liquid fatty phase and/or a water immiscible organic phase of a cosmetic composition, notably in the form of emulsion for caring for and/or making up the skin, the lips and/or superficial body growths.

In various exemplary embodiments, exfoliated phyllosilicates in accordance with the invention can be used to stabilize emulsions, in particular aqueous-phase-in-oil-phase or oil-phase-in-aqueous-phase emulsions.

In the context of the present invention, exemplary exfoliated phyllosilicates need not act as a vehicle for active substances capable of being present in the cosmetic composition. In other words, they are not intended to be employed in the formulation of an active substance into the cosmetic composition.

Various exemplary embodiments of the present invention include processes for making up and/or caring for the skin, lips, nails, and/or superficial body growths comprising the application, to the skin, lips, nails, and/or superficial body growths, of at least one composition in accordance with the present invention.

In various exemplary embodiments, the invention includes a synthetic substrate on which is present, over all or part of its surface, at least one layer of a composition according to the invention.

### DETAILED DESCRIPTION OF EXEMPLARY EMBODIMENTS TRANSLUCENCY AND TRANSPARENCY

Exemplary cosmetic compositions of the invention can have the characteristic, on the one hand, of transparency or translucency of their coat on a make-up substrate and, on the other hand, their own translucent or transparent appearance before application, referred to as “translucent or transparent in the pot” or “bulk translucent or transparent”.

More specifically, the property of transparency or of translucency of exemplary compositions according to the invention can be determined in the following way: the composition to be tested is run into a 30 ml Volga pot, the composition is allowed to cool (for 24 h at ambient temperature) and a white sheet, on which is drawn with a black felt tip pen a cross with a thickness of approximately 2 mm, is placed below. If the cross is visible with the naked eye in daylight at an observation distance of 40 cm, the composition is transparent or translucent.

Exfoliated Phyllosilicates

As defined above, exfoliated phyllosilicates deriving, or else obtained, from at least one intercalated phyllosilicate may be present in exemplary cosmetic compositions according to the invention in various degrees of exfoliation.

Thus, some phyllosilicates may be present in a completely exfoliated form, that is to say in the form of a single sheet, and others, on the other hand, in a partially exfoliated form, that is to say in a form comprising two or more still associated sheets. Advantageously, at least 50%, notably 70%, in particular 90%, indeed even 95% of these exfoliated phyllosilicates may comprise less than 10 sheets, in particular less than 5 sheets, particularly less than 3 sheets, indeed they may even comprise only one sheet of phyllosilicates.

Needless to say that all of these forms may coexist in exemplary cosmetic compositions according to the invention, indeed even mixed with non-exfoliated intercalated phyllosilicates.

Exfoliated phyllosilicates may be obtained by exfoliating intercalated phyllosilicates and then incorporating into a cosmetic composition, or by incorporation of intercalated phyllosilicates into a cosmetic composition which are exfoliated at the time of the preparation of the cosmetic composition.

Exemplary exfoliated phyllosilicates according to the invention can be characterized by an aspect ratio, which is the ratio of the greatest dimension to the smallest dimension. This aspect ratio can vary from 50 to 2000, in particular from 75 to 1500 and especially from 200 to 1000.

Exemplary exfoliated phyllosilicates according to the invention can exhibit a mean thickness of more than 5 Å and a maximum thickness of less than 100 Å and in particular varying from 10 Å to 50 Å.

Due to their small thicknesses, on the scale of a few angstroms, exfoliated phyllosilicates advantageously do not contribute to opaqueness, in contrast to conventional fillers. Like conventional mattifying compositions, exemplary compositions according to the invention, and in particular those intended to be applied to the skin, such as foundation, advantageously prove to be effective in giving a uniform complexion and in particular in softening skin imperfections, while providing a natural appearance to the made-up skin by virtue of the translucency or transparency of their coat. Such compositions have a good surface light-scattering power which is stable over time. In addition, phyllosilicates afford cosmetic compositions with interesting properties in regard of hold and absence of migration, without further affecting their aesthetic qualities such as colored effect and glossiness.

Likewise, phyllosilicates can be used to maintain stabilization of emulsions and, as such, can be used efficiently to totally or partially substitute for and/or replace surfactants generally required to maintain such stabilization.

Exemplary phyllosilicates which can be used according to the invention derive more particularly from the intercalation of mineral clays of smectite type, such as montmorillonites, in particular sodium, potassium and/or calcium montmorillonites, nontronites, beidellites, volkonskoites, hectorites, saponites, sauconites, sobockites, stevensites, svinfordites, vermiculites and their mixtures. Phyllosilicates of montmorillonite type are very particularly suitable for the invention.

Advantageously, exemplary exfoliated phyllosilicates according to the invention have adsorbed at the surface of their sheets, at least one intercalating agent. Said intercalating agent can intercalated between the sheets of the intercalated phyllosilicates which are used to prepare compositions according to the invention. Exemplary exfoliated phyllosilicates have, in particular, at least 15% by weight, in particular at least 20% by weight and more particularly at least 30% by weight of intercalating agent with respect to the weight of dry phyllosilicate (comprising less than 5% of water).

Intercalating agents can be adsorbed over all or part of the surface of the phyllosilicate sheets. Intercalating agents can in particular exhibit, with the phyllosilicate or cations present at its surface, bonds of hydrogen type, ionic or covalent type and/or hydrophilic Van der Waals interactions.

Generally, exemplary intercalating agents according to the invention have, in their chemical structure, at least one hydrophobic group, in particular a C₄ to C₅₀, alkyl, C₄ to C₅₀ alkylene and/or C₄ to C₅₀ alkylaryl chain.

In particular, exemplary exfoliated phyllosilicates according to the invention are advantageously devoid of polyphenol intercalating agents.

In the context of the present invention, preferred intercalating agents include:

oniums having at least one C₁-C₅₀, in particular C₄-C₅₀, hydrocarbonaceous chain; and

intercalating agents “comprising a polar group”, such as organic compounds, of polymeric or nonpolymeric nature, having at least one aromatic ring or at least one polar group chosen from the carbonyl, hydroxyl, polyol (including glycol, glycerol, and the like), carboxylic acid, aldehyde, ketone, amine, amide (linear or cyclic, in particular pyrrolidone or caprolactam), ester, lactone, ether, indeed even sulfate, sulfonate, sulfinate, sulfamate, phosphate, phosphonate and phosphinate groups.

As indicated above, intercalating agents of the onium type can partially or completely replace hydrophilic inorganic ions (Na⁺, K⁺, and the like) present in the phyllosilicates in the natural state. It is also possible to speak of intercalation by ion exchange. With regard to intercalating agents having at least one polar group, they partially or completely replace water molecules which initially coordinated the hydrophilic metal cations present between the sheets.

Intercalating Agent of Onium Type

The term “oniums” can refer to ammonium, sulfonium or phosphonium groups as described, for example, in WO 93/04118 assigned to Allied Signal.

Mention may more particularly be made, among these oniums, of primary ammoniums, secondary ammoniums or tertiary ammoniums, in particular of N⁺H₃R₁, N⁺H₂R₁R₂ and N⁺HR₁R₂R₃ types, and quaternary ammoniums N⁺R₁R₂R₃R4, in which the R₁, R₂, R₃ and R₄ groups, which are identical or different, represent C₁-C₅₀ hydrocarbonaceous chains, at least one of which is more particularly a C₄-C₅₀ chain, it being possible for these hydrocarbonaceous chains to be linear, branched or cyclic and saturated or unsaturated and it being possible for these hydrocarbonaceous chains to comprise one or more heteroatoms, such as O, S, N, Si or P. They can in particular be C₄ to C₅₀ alkyl, C₄ to C₅₀ alkylene or C₄ to C₅₀ alkylaryl chains.

In various exemplary embodiments of the present invention, one of the substituents R₁ to R₄ of the onium group can be of aromatic species (benzyl or phenyl) or arylalkyl, then the other group being alkyl chain C₄ to C₅₀ as defined above.

Mention may in particular be made, by way of illustration and without implied limitation of the intercalating agents of onium type which can be used according to the invention, of those disclosed in WO 93/04118 assigned to Allied Signal, the di- or multionium intercalating agents disclosed in EP 1 038 834 and WO 00/09605 assigned to Amcol, and the intercalating agents comprising an onium group, preferably primary, secondary or tertiary ammoniums having two C≧10 alkyl chains, disclosed in JP 04 357108 assigned to Nippon Paint.

Among these oniums, the primary, secondary, tertiary or quaternary ammoniums, preferably quaternary ammoniums, possessing at least one C₄ to C₁₀ alkyl chain, which chain is preferably linear or branched and preferably saturated, such as, for example, a butyl, isobutyl, pentyl, isopentyl, hexyl, heptyl, 2-ethylhexyl, octyl, nonyl, decyl, undecyl, dodecyl or octadecyl, are very particularly suitable. Among these ammonium cations, those which have at least one alkyl chain of dodecyl or octadecyl type are very particularly suitable.

Intercalating Agents Comprising a Polar Group

The following will be distinguished among intercalating agents comprising a polar group: (a) those which are not of polymeric nature and (b) those which are of polymeric nature.

As used herein, the term “compound of polymeric nature” can refer to a compound having at least two repeat units, in particular at least three repeat units, especially at least ten repeat units, indeed even at least fifteen repeat units. Furthermore, it can be composed of a single repeat unit (homopolymer) or of at least two repeat units of different natures (copolymer).

(a) Intercalating Agents Comprising a Polar Group of Nonpolymeric Nature

They can comprise at least one polar group as defined above and at least preferably one hydrophobic chain, in particular a C₄-C₅₀ hydrocarbonaceous chain, which can be linear, branched or cyclic and saturated or unsaturated and can in addition comprise heteroatoms, such as O, S, N, Si or P. It can in particular be a C₄-C₅₀ alkyl, C₄-C₅₀ alkylene or C₄-C₅₀ alkylaryl chain. Such compounds are disclosed, for example, in the documents U.S. Pat. No. 5,721,306, EP 780 340 and U.S. Pat. No. 6,242,500.

Mention may in particular be made, by way of illustration of this type of compound, of those comprising, as a polar group, at least one group chosen from C₆₋₂₄ alcohol groups, glycerols having at least one C₆-C₂₄ chain, C₆-C₂₄ carboxylic acids, in particular those disclosed in EP 780 340, or amide groups, preferably cyclic amide groups, like lactams, such as pyrrolidone or caprolactam, which are substituted by an aromatic group, such as those disclosed in U.S. Pat. No. 6,242,500, or by a C₄-C₅₀, preferably C₈-C₃₀, preferably C₁₂-C₂₅, alkyl chain, such as, for example, alkylpyrrolidones with a C₄-C₅₀, in particular C₈-C₃₀, alkyl chain. It can in particular be dodecylpyrrolidone, described in particular in Beall G. W., “Nanocomposites produced utilizing a novel dipole clay surface modification in polymer-clay composites,” Chemistry and Technology of Polymer Additives (1999), 266-280, Editor: Al-Malaika, Sahar; Publisher: Blackwell, Oxford, UK.

(b) Intercalating Agents Comprising a Polar Group of Polymeric Nature

They are more particularly synthetic oligomers, homopolymers or copolymers which comprise at least one aromatic ring or one polar group as defined above. The MW can vary from 300 to 200 000, and in particular from 500 to 40 000; such agents are disclosed in particular in U.S. Pat. No. 5,837,763.

These oligomers or polymers can be hydrophilic or hydrophobic.

Mention may in particular be made, by way of representation of these hydrophilic polymeric intercalating agents, of polyvinylpyrrolidone (PVP) derivatives, polyvinyl alcohol (PVA) derivatives, in particular when the latter are virtually in their polyvinyl acetate hydrolysed form or, in other words, have less than 5% of residual acetyl groups, polyacrylic derivatives in their polymeric and copolymeric form and more particularly in the form of their metal salts, polymethacrylic acid (PMAA) derivatives, polyvinyloxazolidone (PVO) and polyvinylmethyloxazolidone (PVMO) derivatives, or polyvinyloxazoline derivatives.

Mention may also be made, as other exemplary hydrophilic polymeric intercalating agents, of copolymers of the above mentioned units, copolymers between these same units or copolymers with other hydrophilic or strongly polar monomers, such as: hydroxyethyl (meth)acrylate, 2-hydroxypropyl (meth)acrylate, methyl (meth)acrylate, vinyl acetate, (meth)acrylamide, N,N-dimethylacrylamide, crotonic acid, maleic anhydride or methyl vinyl ether.

Polymeric intercalating agents can also be organophilic or even lipophilic, provided that they have at least one, indeed even more, polar group(s) as defined above. Copolymers obtained by reaction between a polar and hydrophilic monomer as chosen above (vinylpyrrolidone, vinyloxazoline, vinyloxazolidone, vinyl alcohol or (meth)acrylic acid with at least one more organophilic, indeed lipophilic, monomer, such as (meth)acrylic acid esters, such as ethyl, butyl, isobutyl, tert-butyl, hexyl, cyclohexyl, octyl, 2-ethylhexyl, nonyl, decyl, undecyl, dodecyl, octadecyl or behenyl (meth)acrylates, vinyl esters, such as propionate, versatate or benzoate; (meth)acrylamides, such as diacetone acrylamide, butyl(meth)acrylamide, tert-butyl(meth)acrylamide, tert-hexyl(meth)acrylamide or tert-octyl(meth)acrylamide; or olefins, such as ethylene, propylene, butene, isobutene, hexene, octene, dodecene, octadecene, icosene, styrene and substituted styrenes, can be selected in particular.

Other water-soluble or hydrophilic polyhydric alcohols and polymeric polyols, such as polysaccharides, are also capable of constituting polymeric intercalating agents.

Some phyllosilicates/intercalating agent combinations, such as montmorillonite-PVP, montmorillonite-PVA and montmorillonite-alkylpyrrolidone, such as, for example, montmorillonite-dodecylpyrrolidone, can be particularly advantageous within the scope of the invention.

Intercalation of phyllosilicates, considered in the context of the present invention, can be carried out according to conventional protocols, such as those disclosed in particular in U.S. Pat. No. 5,721,306 and WO 93/04118.

To be specific, intercalating agents can be introduced or adsorbed inside the interlayer spaces of the phyllosilicate according to the following exemplary embodiment: the intercalation is carried out by intimately mixing the phyllosilicates by extrusion or stirring with a propeller mixer, so as to form an intercalating composition comprising the phyllosilicate in an intercalating polymer, an aqueous solution of intercalating agent or an organic solution of intercalating agent. In order to obtain sufficient intercalation for the purposes of exfoliation, the intercalating agent is generally brought into contact with the phyllosilicate in the intercalation composition in a proportion of an intercalating agent/phyllosilicate ratio by weight of at least approximately 1/20, in particular of at least approximately 1/10 and more particularly of approximately 1/5, indeed even of approximately 1/4, so as to obtain effective intercalation of the agent between adjacent sheets of phyllosilicate. An interlayer space can thus be increased by 10 to 100 angstroms to guarantee easy and complete resulting exfoliation. An intercalation vehicle, preferably water, if appropriate as a mixture with an organic solvent, can be introduced after pre-dissolving or—dispersing the intercalating agent in the vehicle, or can be directly mixed with the dry intercalating agent and the dry phyllosilicate. Intercalating agent is generally present in a proportion of at least 15% by weight, in particular of at least 20% by weight and more particularly of at least 30% by weight with respect to the weight of dry phyllosilicate (comprising less than 5% of water). This amount can vary in particular from 20 to 50% by weight with respect to the weight of phyllosilicate in the dry state.

Polymeric intercalating agents may often be obtained by direct polymerization (homo or copolymerisation) of monomers which were previously intercalated between the sheets of phyllosilicates.

As regards exfoliation of the phyllosilicates thus intercalated, it can be carried out conventionally, generally by application, to the medium in which the intercalated phyllosilicates are dispersed, of a shear rate sufficient to bring about the desired delamination. This aspect is expanded upon more particularly below.

Furthermore, a number of intercalated and exfoliated phyllosilicates are already available commercially.

Mention may more particularly be made, by way of illustration of the exfoliated phyllosilicates which are suitable for the invention, of those sold by:

Nanocor in the United States. Mention may be made, as examples of clays (montmorillonite type) intercalated by a quaternary alkylammonium, that is to say by ion exchange, of Nanomer I.24 T, Nanomer I.30 TC and Nanomer I.34 TCN; as examples of clays intercalated by an organic pyrrolidone derivative (technology of the following type: ion-dipole by exchange of the coordinated water), of Nanomer 1.35 K and Nanomer 1.46 D, also described in: Lan, T., “Advances in nanomer additives for clay/polymer nanocomposites” (Nanocor), Additives 99, International Conference, 8th, San Francisco, Mar. 22-24, 1999 (1999), Paper 12/1— Paper 12/11, Publisher: Executive Conference Management, Plymouth, Mich.; and

Southern Clay Products in the United States, which also produces clays intercalated by a quaternary alkylammonium, e.g. Cloisite 25A and Cloisite 30B, cited in the article by Poittevin B., Polymer 43, 4017-23 (2002).

Exemplary exfoliated phyllosilicates according to the invention are preferably present in an amount which is effective in texturizing or alternatively in stiffening or gelling the liquid fatty phase or water-insoluble organic phase of the composition.

In particular, exfoliated phyllosilicates can be present in a proportion of 0.05 to 30% by weight, in particular of 0.1 to 20% by weight and more particularly of 0.5 to 10% by weight in the cosmetic composition.

To the extent, exfoliated clays are used for the purpose of stabilizating emulsion, an efficient amount is, evidently, liable to significantly vary whether or not those clays are associated with other surfactants. A skilled artisan will be capable of proceeding with corresponding adjustments.

Phyllosilicates are generally introduced directly in an exfoliated form into the composition. However, the invention also applies to the compositions in which the exfoliated phyllosilicates would be generated in situ, for example by simple stirring of the cosmetic composition comprising intercalated phyllosilicates, that is to say in a nonexfoliated form, or shaking of the package containing the composition. As specified above, the exfoliated phyllosilicates according to the invention can be present as a mixture with non-exfoliated intercalated phyllosilicates which are identical or different in chemical nature.

Fatty Phase

Various exemplary embodiments of the compositions according to the invention can comprise at least one liquid fatty phase. In particular, the fatty phase can constitute the continuous phase or the dispersed phase of an emulsion and in particular of a microemulsion.

Exemplary compositions can have, for example, a continuous fatty phase which can comprise less than 10% by weight of water, in particular less than 5% by weight of water, indeed even less than 1% by weight of water with respect to its total weight and in particular can be in the anhydrous form.

The term “liquid fatty phase” can refer, as used herein, to a fatty phase which is liquid at ambient temperature (25° C.) and at atmospheric pressure composed of one or more fatty substances which are liquid at ambient temperature of oil type which are compatible with one another.

The term “structured liquid fatty phase” can refer to a stiffened or gelled liquid fatty phase or only thickened.

The term “stiffened liquid fatty phase” can refer to a fatty phase that does not flow under the effect of its own weight when combined with an exfoliated phyllosilicate according to the invention.

The term “gelled or thickened liquid fatty phase” can refer to a fatty phase in which viscosity is increased because of its combination with an exfoliated phyllosilicate according to the invention.

In various exemplary embodiments, the fatty phase of the composition according to the invention can in particular comprise, as liquid fatty substance, at least one volatile or nonvolatile oil or one of their mixtures.

In various exemplary embodiments, the oily phase of the composition according to the invention may be present in a proportion varying from 1 to 80%, in particular from 1 to 50% by weight relative to the total weight of the composition.

The term “volatile oil” can refer, as used herein, to any oil capable of evaporating on contact with the skin in less than one hour, at ambient temperature and atmospheric pressure. Exemplary volatile oils of the invention are volatile cosmetic oils which are liquid at ambient temperature having a non zero vapour pressure, at ambient temperature and atmospheric pressure, ranging in particular from 0.01 to 300 mm of Hg (1.33 Pa to 40 000 Pa) and preferably greater than 0.3 mm of Hg (30 Pa).

The term “nonvolatile oil” can refer to an oil which remains on the skin at ambient temperature and atmospheric pressure for at least several hours and which has in particular a vapour pressure of less than 0.01 mm of Hg (1.33 Pa).

Volatile or nonvolatile oils can be hydrocarbonaceous oils, in particular of animal or vegetable origin, silicone oils, or their mixtures. The term “hydrocarbonaceous oil” can refer to an oil comprising mainly hydrogen and carbon atoms and optionally oxygen, nitrogen, sulfur and/or phosphorus atoms.

Volatile hydrocarbonaceous oils can be chosen from hydrocarbonaceous oils having from 8 to 16 carbon atoms and in particular branched C₈-C₁₆ alkanes, such as C₈-C₁₆ isoalkanes of petroleum origin (also known as isoparaffins), such as isododecane (also known as 2,2,4,4,6-pentamethylheptane), isodecane or isohexadecane, and, for example, the oils sold under the trade names of Isopars® or Permetyls®, branched C₈-C₁₆ esters, such as isohexyl neopentanoate, and their mixtures. Other volatile hydrocarbonaceous oils, such as petroleum distillates, in particular those sold under the name Shell Solt® by Shell, can also used.

Use may also be made, as volatile oils, of volatile silicones, such as, for example, volatile linear or cyclic silicone oils, in particular those having a viscosity ≦8 centistokes (8×10⁻⁶ m²/s), and having in particular from 2 to 7 silicon atoms, these silicones optionally comprising alkyl or alkoxy groups having from 1 to 10 carbon atoms. Mention may in particular be made, as volatile silicone oil which can be used in the invention, of octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane, dodecamethylpentasiloxane and their mixtures.

Volatile oils can be present in the fatty phase according to the invention at a content ranging from 0.1% to 98% by weight, in particular from 1% to 65% by weight and especially from 2% to 50% by weight with respect to the total weight of the fatty phase.

Nonvolatile oils can in particular be chosen from nonvolatile hydrocarbonaceous oils, if appropriate fluorinated, and/or nonvolatile silicone oils.

Mention may in particular be made, as exemplary nonvolatile hydrocarbonaceous oils, of:

hydrocarbonaceous oils of animal origin;

hydrocarbonaceous oils of vegetable origin, such as triglycerides composed of esters of fatty acids and of glycerol, the fatty acids of which can have varied chain lengths from C₄ to C₂₄, it being possible for these chains to be linear or branched and saturated or unsaturated; these oils are in particular wheat germ, sunflower, grape seed, sesame, maize, apricot, castor, avocado, olive, soybean, sweet almond, palm, rapeseed, cotton seed, hazelnut, macadamia, karite, jojoba, alfalfa, poppy, pumpkinseed, sesame, cucumber, blackcurrant, evening primrose, millet, barley, quinoa, rye, safflower, candlenut, passion flower or musk rose oil; karite butter; or triglycerides of caprylic/capric acids, such as those sold by Stéarineries Dubois or those sold under the names Miglyol 810®, 812® and 818® by Dynamit Nobel;

synthetic ethers having from 10 to 40 carbon atoms;

linear or branched hydrocarbons of mineral or synthetic origin, such as liquid petrolatum, polydecenes, hydrogenated polyisobutene, such as parleam, squalane and their mixtures;

synthetic esters, such as oils of formula R₁COOR₂ in which R₁ represents the residue of a linear or branched fatty acid comprising from 1 to 40 carbon atoms and R₂ represents a hydrocarbonaceous chain, in particular a branched hydrocarbonaceous chain, comprising from 1 to 40 carbon atoms, provided that R₁+R₂ is ≧10, such as, for example, purcellin oil (cetearyl octanoate), isopropyl myristate, isopropyl palmitate, C₁₂ to C₁₅ alkyl benzoates, hexyl laurate, diisopropyl adipate, isononyl isononanoate, 2-ethylhexyl palmitate, isostearyl isostearate or heptanoates, octanoates, decanoates or ricinoleates of alcohols or of polyalcohols, such as propylene glycol dioctanoate; hydroxylated esters, such as isostearyl lactate or diisostearyl malate; polyol esters and pentaerythritol esters;

fatty alcohols which are liquid at ambient temperature comprising a branched and/or unsaturated carbonaceous chain having 12 to 26 carbon atoms, such as octyldodecanol, isostearyl alcohol, oleyl alcohol, 2-hexyldecanol, 2-butyloctanol and 2-undecylpentadecanol;

higher fatty acids, such as oleic acid, linoleic acid or linolenic acid; and

silicone oils of polymethylsiloxane (PDMS) type and their mixtures.

Exemplary nonvolatile silicone oils which can be used in the composition according to the invention can be nonvolatile polydimethylsiloxanes (PDMSs), polydimethylsiloxanes comprising pendant alkyl or alkoxy groups and/or alkyl or alkoxy groups at the silicone chain ends, groups each having from 2 to 24 carbon atoms, or phenylated silicones, such as phenyl trimethicones, phenyl dimethicones, phenyltrimethylsiloxydiphenylsiloxanes, diphenyl dimethicones, diphenylmethyldiphenyltrisiloxanes and (2-phenylethyl)trimethylsiloxysilicates.

Nonvolatile oils can be present in the liquid fatty phase in a content ranging from 0.01% to 90% by weight, in particular from 0.1% to 85% by weight and especially from 1% to 70% by weight with respect to the total weight of the phase.

More generally, an exemplary fatty phase which is liquid at ambient temperature and at atmospheric pressure can be present in a proportion of 0.01 to 99.5% by weight, in particular from 0.5 to 85% by weight, especially from 10 to 80% by weight, indeed even from 20 to 75% by weight with respect to the weight of the composition.

Exemplary embodiments of compositions according to the invention can also comprise a fatty substance which is solid at ambient temperature and at atmospheric pressure, such as waxes, pasty fatty substances, gums and their mixtures, with a proviso, of course, that the latter do not affect its translucency or transparency or those of its coat on the make-up substrate.

As regards fatty substances which are solid at ambient temperature and at atmospheric pressure, they can be chosen from waxes, pasty fatty substances, gums and their mixtures. As specified above, this solid fatty substance can only be present in an amount compatible with the translucent or transparent nature required according to the invention.

Exemplary waxes can be solid at ambient temperature (25° C.), with a reversible solid/liquid change in state, having a melting point of greater than 30° C. which can range up to 200° C., having a hardness of greater than 0.5 MPa and exhibiting, in a solid state, an anisotropic crystalline arrangement. They can be hydrocarbonaceous, fluorinated and/or silicone waxes and can be of animal, vegetable, mineral or synthetic origin. They can be chosen, for example, from beeswax, carnauba wax, candelilla wax, paraffin waxes, hydrogenated castor oil, silicone waxes, microcrystalline waxes and their mixtures.

The term “pasty” as used herein refers to a lipophilic fatty compound with a reversible solid/liquid change of state exhibiting in the solid state, an anisothropic crystalline arrangement, and comprising, at a temperature of 23° C., a liquid fraction and a solid fraction.

The term “pasty compound” as used herein refers to a compound having a hardness, at 20° C., ranging from 0.001 to 0.5 MPa, in particular from 0.002 to 0.4 MPa.

Hardness is measured according to a method including penetrating a probe into a sample of compound and in particular using a texture analyzer (for example, TA-XT2i from Rheo) equipped with a stainless steel cylinder with diameter 2 mm. The hardness measurement can be carried out at 20° C. at the center of 5 samples. The cylinder is introduced into each sample at a pre-rate of 1 mm/s and then at a measuring rate of 0.1 mm/s, the total displacement being 0.3 mm. The recorded hardness value is that of the maximum peak observed.

Further, at a temperature of 23° C., the pasty compound can be in the form of a liquid fraction and a solid fraction. In other words, the starting melting temperature of the pasty compound is less than 23° C. The liquid fraction of the pasty compound measured at 23° C., represents 9 to 97% by weight relative to the total weight of the compound. This liquid fraction at 23° C. represents, in particular, from 15 to 85%, particularly from 40 to 85% by weight relative to the total weight of the compound.

The liquid fraction by weight of the pasty compound at 23° C. can be equal to the ratio of the enthalpy of fusion consumed at 23° C. to the enthalpy of fusion of the pasty compound.

Enthalpy of fusion of the pasty compound is the enthalphy consumed by the compound to change from the solid state to the liquid state. The pasty compound is in “solid state” when the whole of its mass is in a cristalline solid form. The pasty compound is in “liquid state” when the whole of its mass is in a liquid form.

Enthalpy of fusion of the pasty compound is equal to the area under the curve of the termogram obtained using a differential scanning calorimeter (D.S.C), such as the MDSC 2920 calorimeter sold by TA Instrument, with rise in temperature of 5 or 10° C. to minute, according to the ISO standard 11357-3:1999. The enthalpy of fusion the pasty compound is the amount of energy required to change the compound from the solid state to the liquid state. It is expressed in J/g.

The enthalpy of fusion consumed at 23° C. is the amount of energy absorbed by the sample to change from the solid state to the state which it exhibits at 23° C., composed of a liquid fraction and a solid fraction.

In exemplary embodiments, the liquid fraction of the pasty compound measured at 32° C. can be, in particular, from 30 to 100% by weight of the compound, particularly from 80 to 100%, more particularly from 90 to 100% by weight of the compound. To the extent the liquid fraction of the pasty compound measured at 32° C. is equal to 100%, the temperature of the end of the melting range of the pasty compound is less than or equal to 32° C.

The liquid fraction of the pasty compound measured at 32° C. can be equal to the ratio of the enthalpy of fusion consumed at 32° C. to the enthalpy of fusion of the pasty compound. The enthalpy of fusion consumed at 32° C. is computed in the same way as the enthalphy of fusion consumed at 23° C.

More particularly, these fatty substances can be hydrocarbonaceous compounds, optionally of polymeric type; they can also be chosen from silicone compounds; they can also be provided in the form of a mixture of hydrocarbonaceous and/or silicone compounds. In the case of a mixture of various pasty fatty substances, use is predominantly made preferably of pasty hydrocarbonaceous compounds (comprising mainly carbon and hydrogen atoms and optionally ester groups).

Mention may be made, among the pasty compounds capable of being used in the composition according to the invention, of lanolins and lanolin derivatives, such as acetylated lanolins, oxypropylenated lanolins or isopropyl lanolate, and their mixtures. Use may also be made of esters of fatty acids or of fatty alcohols, in particular those having 20 to 65 carbon atoms, such as triisostearyl or cetyl citrate; arachidyl propionate; poly(vinyl laurate); cholesterol esters, such as triglycerides of vegetable origin, for example hydrogenated vegetable oils, viscous polyesters and their mixtures. Use may be made, as triglyceride of vegetable origin, of hydrogenated castor oil derivatives, such as “Thixinr®” from Rheox.

Mention may also be made of silicone pasty fatty substances, such as polydimethylsiloxanes (PDMSs) with high molecular weights and in particular those having pendant chains of the alkyl or alkoxy type having from 8 to 24 carbon atoms and a melting point of 20-55° C., such as stearyl dimethicones, in particular those sold by Dow Corning under the trade names DC2503® and DC25514®, and their mixtures.

Water-Immiscible Organic Phase

Exemplary embodiments of the composition according to the invention can comprise a water-immiscible organic phase. This immiscible organic phase can be composed of one or more water-immiscible organic solvents.

As used herein, a solvent can be regarded as water-immiscible from the moment when it displays a miscibility in water of less than or equal to 50% by weight, in particular of less than or equal to 30% by weight, indeed even of less than or equal to 20% by weight and more particularly of less than or equal to 10% by weight, indeed even of less than or equal to 9% by weight at 25° C.

Mention may more particularly be made, by way of representation of these organic solvents which are volatile or nonvolatile at ambient temperature, of short-chain esters (having from 4 to 8 carbon atoms in total), such as ethyl acetate, propyl, butyl or aryl acetates, isopentyl acetate and their mixtures.

Aqueous Phase

Exemplary embodiments of the composition according to the invention can also comprise an aqueous medium, constituting an aqueous phase, which can form the continuous phase of the composition.

The aqueous phase can be composed essentially of water. It can also comprise a mixture of water and of a water-miscible organic solvent (miscibility in water of greater than 50% by weight at 25° C.), such as low monoalcohols having from 1 to 5 carbon atoms, for example ethanol or isopropanol, glycols having from 2 to 8 carbon atoms, for example propylene glycol, ethylene glycol, 1,3-butylene glycol or dipropylene glycol, C₃-C₄ ketones, C₂-C₄ aldehydes and their mixtures.

In exemplary embodiments, the aqueous phase (water and optionally the water-miscible organic solvent) can be present at a content ranging from 1 to 95% by weight, in particular ranging from 3 to 80% by weight and especially ranging from 5 to 60% by weight with respect to the total weight of the composition. According to a particular embodiment, the aqueous phase can be present in an amount less than 50% by weight and more particularly less than 20% by weight relative to the weight of the composition.

Emulsion

As used herein, the term “emulsion” can refer to an immiscible two-liquid system in which one is finely divided in droplets into the other. The dispersed phase is also known as “internal or discontinuous phase”. The dispersing phase is also known as “continuous or external phase”. Emulsions into which the dispersed phase is lipophilic, such as a vegetable or mineral oil, and the dispersing phase is hydrophilic, such as water, are known as “aqueous emulsions” (O/W: oil in water). Emulsions into which the dispersed phase is hydrophilic and the dispersing phase is lipophilic are known as “oily emulsions” (W/O: water in oil). Multiple emulsions are also known, such as W/O/W (water in oil in water).

In exemplary embodiments of the present invention, emulsions can comprise a lipophilic and a hydrophilic phase, the latter not necessarily being water.

In particular, a composition in a form of an emulsion may be transparent or translucent and/or may be capable of giving a transparent or translucent coat.

According to one of its embodiments, the composition comprises at least one emulsifying agent and, if appropriate, at least one co-emulsifying agent in an amount lower than 30%, in particular lower than 20%, particularly lower than 10%.

According to another of its embodiments, the composition comprises at least one emulsifying agent and, if appropriate, at least one co-emulsifying agent in an amount ranging from 0.2 to 30% by weight relative to the total weight of the composition, in particular from 0.3 to 20% by weight and advantageously from 0.5 to 15% by weight relative to the total weight of the composition.

In exemplary embodiments, the composition comprises less than 0.5% by weight relative to the total weight of the composition of emulsifying and co-emulsifying agent.

In exemplary embodiments, the composition according to the invention in a form of an emulsion is advantageously free from any emulsifying agent allowing its stabilization, to the exception of the exfoliated phyllosilicate in accordance with the invention.

The skilled artisan readily knows how to select, for a composition comprising selected aqueous phase and fatty phase, in a selected ratio, the emulsifying agent and possibly the co-emulsifying agent from the prior art and their respective proportions for obtaining a stable emulsion.

The term “stable emulsion” as used herein refers to an emulsion which, when placed in a transparent container, itself placed in an incubator at 45° C. for two months, does not undergo a separation of phases (or exudate) at the end of this period. The separation of phases (or exudation) is visually detected through the transparent wall of the container, when it is withdrawn from the incubator.

In various exemplary embodiments, the compositions may, notably be in the form of emulsions comprising an aqueous phase and an oily phase, dispersed one in another, for example in the form of emulsions water-in-oil (W/O) or oil-in-water (O/W) or multiple emulsions (W/O/W or O/W/0) or in the form of emulsions chosen among the usual emulsions or particular emulsions such as:

emulsions O/W comprising oily globules comprising a lamellar liquid crystal coating, such as described in EP-A-641 557 and EP-A-705 593;

J emulsions O/W without emulsifying agent, stabilized with hydrodispersible anionic polymer, such as desribed in EP-A-864 320;

emulsions O/W comprising polymers derived from sulfonic 2-acrylamido-2-methylpropane acid (AMPS polymer), such as described in EP-A-815 844;

emulsions O/W stabilized with hydrophobic AMPS polymers, such as described in EP-A-1 069 142, WO-A-2002/43689, WO-A-2002/44231, WO-A-2002/44271, WO-A-2002/44270, WO-A-2002/43686, WO-A-2002/44267, WO-A-2002/43688, WO-A-2002/43677, WO-A-2002/43687, WO-A-2002/44230;

fluid emulsions comprising thermo associative polymers, such as described in EP-A-1 355 990, EP-A-1 355 625, EP-A-1 307 501, EP-A-1 363 954;

emulsions O/W obtained according to the PIT process (emulsion obtained with phase inversions, PIT: phase inversion temperature), such as described in WO-A-89/11907, DE-A-431 8171, and EP-A-815 846; and

nanoemulsions such as described in the applications EP-A-728 460, EP-A-780 114, EP-A-780 115, EP-A-879 789, EP-A-1 010 413, EP-A-1 010 414, EP-A-1 010 415, EP-A-1 010 416, EP-A-1 013 338, EP-A-1 016 453, EP-A-1 018 363, EP-A-1 020 219, EP-A-1 025 898, EP-A-1 120 102, EP-A-1 120 101, EP-A-1 160 005, EP-A-1 172 077 and EP-A-1 353 629.

In exemplary embodiments, the proportion of oily phase in an emulsion may range from 1 to 80% by weight, and in particular from 1 to 50% by weight relative to the total weight of the composition. Oils, emulsifying agents and co-emulsifying agents possibly present in the composition in the form of emulsions can be chosen among those which are usually used in the cosmetic or dermatologic field. Emulsifying agents and co-emulsifying agent when present, are generally in a proportion ranging from 0.2 to 30% by weight, in particular from 0.3 to 20% by weight, and more particularly from 0.5 to 15% by weight relative to the total weight of the composition. Further, emulsions may comprise lipidic vesicles.

In exemplary embodiments, the emulsifying agent may be chosen from the group consisting of amphoteric, anionic, cationic or non ionic emulsifying agents, alone or in combination. Emulsifying agents are chosen in an appropriate way according to the continuous phase of the emulsion to be obtained (W/O or O/W). To the extent an emulsion is in multiple form, it usually comprises an emulsifying agent in the primary emulsion and an emulsifying agent in the external phase into which is introduced the primary emulsion.

Mention may be made, as emulsifying agents which can be used in the preparation of a W/O emulsion, of sorbitan or glycerol or glucid ether or alkylester; the silicone-sufactants such as dimethicone copolyol such as the mixture of dimethicone copolyol and cyclomethicones sold as DC 5225C and DC 3225C by Dow Corning, and as alkyldimethicone copolyol such as Laurylmethicone copolyol sold as “Dow Corning 5200 formulation Aid” by Dow Corning, cetyl dimethicone copolyol sold as “Abil EM 90” by Goldschmidt and the mixture of polyglyceryl-4 isostearate/cetyl dimethicone copolyol/hexyl laurate sold as “Abil WE09®” by Goldschmidt. One or more co-emulsifying agent(s) may be added which may be advantageously chosen in the group consisting of polyols branched chain fatty acid esters, and in particular sorbitan and/or glycerol branched chain fatty acid esters, as for example, polyglyceryl isostearate, such as the product Isolan GI34 commercialized by Goldschmidt, sorbitan isostearate, such as the product ARLACEL 987 commercialized by ICI, sorbitan and glycerol isostearate, such as the product ARLACEL 986 commercialized by ICI, and their mixtures.

Mention may be made, as emulsifying agents which may be used in the preparation of O/W emulsion, of non ionic emulsifying agents such as oxyalkylenated polyol fatty acid esters (in particular polyoxyethylenated) and for example the glycol polyethylene stearate such as PEG-100 stearate, PEG-50 stearate and PEG-40 stearate; and their mixtures such as the mixtures of glyceryl monostearate and glycol polyethylene stearate (1000E) commercialized as SIMULSOL 165 by SEPPIC; oxylalkylenated sorbitan fatty acid ester comprising for example from 20 to 1000E, and for example those commercialized as TWEEN 20 or TWEEN 60 from UBIQEMA; the oxyalkylenated fatty alcohol ether (oxyethylenated and/or oxypropylenated); alkoxylated or not glucid ester, as sucrose stearate and as sesqui stearate methylglucose PEG 20; sorbitan esters such as sorbitan palmitate commercialized as SPAN 40 by UBIQEMA; fatty alcohol diacid esters, such as dimyristyltartrate; the mixtures of those emulsifying agents such as for example the mixture of glycerylstearate and PEG-100 stearate, commercialized as ARLACEL 165 by UNIQEMA; and the mixtures comprising emulsifying agents, such as the mixtures comprising dimyristyltartrate, cetearylic alcohol Pareth-7, and PEG-25 laureth-25, sold as COSMACOL PSE from SASOL (named as CTFA: dimyristyltartrate/cetearyl alcohol/12-15 Pareth7/PPG 25 laureth 25).

Co-emulsifying agents such as for example fatty alcohols in C₈ to C₂₆, as for example cetylic alcohol, stearylic alcohol and their mixtures (cetearylic alcohol), dodecanoloctyl, butyloctanol-2, hexyldecanol-2, undecylpentadecanole-2 or oleic alcohol, or fatty acid may be added to emulsifying agents.

Emulsions may also be prepared without any emulsifying surfactant or comprising less than 0.5% of such agent by weight relative to the total weight of the composition, by using appropriate compounds, such as, for example, polymers having emulsifying properties such as polymers commercialized as CARBOPOL 1342 and PEMULEN from Noveon; or polymers in emulsion such as SEPIGEL 305 from SEPPIC (INCI: polyacrylamid/C₁₃-C₁₄ isoparaffin/laureth-7); particles of ionic or non ionic polymers, more particularly particles of anionic polymers such as, in particular, isophtalic acid polymers or sulfoisophtalic acid, and in particular phtalate/sulfoisophtalate/glycol (for example diethylene glycol)/phtalate/isophtalate/1,4-cyclohexane-dimethanol copolymer, INCI: diglyco/CHDM/isophtalate/SIP copolymer) sold as EASTMAN AQ POLYMER (AQ ³⁵S, AQ 38S, AQ 55S, AQ 48 ULTRA) from Eastman Chemical. Emulsions without emulsifying agent may be also prepared which are stabilized with silicone particles or metal oxide particles such as TiO₂ or others.

Film-Forming Polymers

In various exemplary embodiments, compositions according to the invention can additionally comprise at least one film-forming polymer, with a proviso that it does not affect the transparency or translucency required according to the invention. The film-forming polymers which are provided in a dissolved form, generally dissolved in the continuous phase of the composition, are more particularly suitable as such.

However, film-forming polymers in dispersed form may also be suitable, especially if the size of the reduced particles is less than or equal to 60 nm, and a low polydispersity is satisfied.

As used herein, the term “film-forming polymer” refers to a polymer capable of forming, by itself alone or in the presence of an additional agent which is able to form a film, a continuous film on a substrate.

In order to retain the character of transparency or translucency of the compositions according to the invention, the use of polymers in the dissolved form is prefered.

In exemplary embodiments, fat-soluble polymers are employed, in particular in the case where the cosmetic composition comprises at least one liquid fatty phase.

Fat-soluble polymers can be of any chemical nature and encompass in particular:

A. fat-soluble and amorphous homopolymers and copolymers of olefins, of cycloolefins, of butadiene, of isoprene, of styrene, of vinylethers, esters or amides, or of (meth)acrylic acid esters or amides comprising a linear, branched or cyclic C₄₋₅₀ alkyl group, and preferably amorphous.

Mention may be made, as specific fat-soluble copolymers, of:

(i) acrylic/silicone grafted polymers comprising a silicone backbone and acrylic grafts or comprising an acrylic backbone and silicone grafts, such as the product sold under the name SA 70.5 by 3M and those disclosed in U.S. Pat. No. 5,725,882, U.S. Pat. No. 5,209,924, U.S. Pat. No. 4,972,037, U.S. Pat. No. 4,981,903, U.S. Pat. No. 4,981,902, U.S. Pat. No. 5,468,477, U.S. Pat. No. 5,219,560 and EP 0 388 582.

(ii) fat-soluble polymers carrying fluorinated groups belonging to one of the classes described above, in particular those disclosed in U.S. Pat. No. 5,948,393, or the alkyl (meth)acrylate/perfluoroalkyl (meth)acrylate copolymers disclosed in EP 0 815 836 and U.S. Pat. No. 5,849,318.

(iii) polymers or copolymers resulting from the polymerization or from the copolymerization of an ethylenic monomer comprising one or more ethylene units, preferably conjugated units (or dienes). Use may be made, as polymers or copolymers resulting from the polymerization or from the copolymerization of an ethylenic monomer, of vinyl, acrylic or methacrylic copolymers which can be block copolymers, such as diblock or triblock copolymers, or even multiblock copolymers comprising varied forms. The film-forming agent comprising at least one ethylenic unit can comprise, for example, a styrene (S) block, an alkylstyrene (AS) block, an ethylene/butylene (EB) block, an ethylene/propylene (EP) block, a butadiene (B) block, an isoprene (I) block, an acrylate (A) block, a methacrylate (MA) block or a combination of these blocks.

(iv) copolymers of N-vinylpyrrolidone and of olefins, the number of carbon atoms in the olefins being >8, e.g. N-vinylpyrrolidone/hexadecene or N-vinyl-pyrrolidone/icosene copolymer.

(v) homo- or copolymers of vinyl esters which are fat-soluble, such as poly(vinyl laurate)s, poly(vinyl stearate) and their copolymers with vinyl acetate.

(vi) homo- or copolymers of (meth)acrylic esters or amides. The methacrylic ester monomers then result from esterification of (meth)acrylic acid with an alcohol having a number of carbon atoms of >4 and preferably of ≧8, with as example 2-ethylhexyl (meth)acrylate, n-octyl (meth)acrylate, dodecyl (meth)acrylate, stearyl (meth)acrylate or behenyl (meth)acrylate. These long-chain (meth)acrylates can be copolymerized with other (meth)acrylic esters, vinyl esters or styrene.

B. amorphous fat-soluble polycondensates in particular not comprising hydrogen bonds denoting groups, especially polyesters comprising C₄₋₅₀ alkyl side chains, polyesters resulting from the condensation of fatty acid dimers or polyesters comprising a silicone segment in the form of a block, graft or end group, which are solid at ambient temperature,

C. fat-soluble and amorphous polysaccharides comprising alkyl (ether or ester) side chains, especially alkylcelluloses carrying linear or branched and saturated or unsaturated C₁ to C₈ alkyl radicals, such as ethylcellulose and propylcellulose.

Generally, exemplary film-forming fat-soluble polymers capable of being employed according to the invention can have a molecular weight of between 1000 and 500 000, preferably between 2000 and 250 000, and a glass transition temperature of between −100° C. and +300° C., in particular between −50° C. and +100° C. and especially between −10° C. and +90° C.

In the specific case of cosmetic compositions comprising at least one water-immiscible organic phase and in particular of nail varnish type, nitrocelluloses and/or cellulose esters, such as cellulose acetates, cellulose propionates, cellulose butyrates, cellulose acetate/propionates, cellulose acetate/butyrates and their mixtures are very particularly suitable as film-forming agents.

Exemplary polymers in compositions according to the invention can also be used in an amount varying from 0.01% to 40% with respect to the total weight of the composition, in particular from 1% to 20%, such as, for example, from 1% to 10%.

Film-forming polymers can be used in combination with auxiliary agents which are able to form a film. Such an agent which is able to form a film can be chosen from any compound known to a person skilled in the art as being capable of fulfilling the desired role and can in particular be chosen from plasticizing agents and coalescence agents.

Coloring Agent

As specified above, exemplary compositions according to the invention can be colored.

In various exemplary embodiments, compositions according to the invention can in particular comprise coloring agents chosen from water-soluble dyes, fat-soluble dyes, pigments and lakes.

The term “pigments” should be understood as comprising white or colored and inorganic or organic particles which are insoluble in the liquid hydrophilic phase and which are intended to color and/or opacify the composition. The term “fillers” should be understood as comprising colorless or white, inorganic or synthetic and lamellar or nonlamellar particles. The term “pearlescent agents” should be understood as comprising iridescent particles produced in particular by certain shellfish in their shells or else synthesized.

Mention maybe made, as exemplary inorganic pigments which can be used in the invention, of titanium, zirconium or cerium oxides and zinc, iron or chromium oxides, ferric blue, manganese violet, ultramarine blue and chromium hydrate. Mention may be made, among exemplary organic pigments which can be used in the invention, of carbon black, pigments of D & C type and lakes based on cochineal carmine or on barium, strontium, calcium or aluminium, or the diketopyrrolopyrroles (DPPs) disclosed in EP-A-542669, EP-A-787730, EP-A-787731 and WO-A-96/08537. The amount and/or the choice of pigments are generally adjusted by taking into account the amount of exfoliated intercalated phyllosilicates present in the cosmetic composition under consideration.

Pearlescent agents can be present in the composition in a proportion of 0.01 to 25% by weight.

Exemplary pearlescent pigments can be chosen from white pearlescent pigments, such as mica covered with titanium or with bismuth oxychloride, colored pearlescent pigments, such as titanium mica with iron oxides, titanium mica with in particular ferric blue or chromium oxide, or titanium mica with an organic pigment of the abovementioned type, and pearlescent pigments based on bismuth oxychloride.

Exemplary fat-soluble dyes include, for example, Sudan red III (CTFA: D & C Red 17), lutine, quinizarin green (CTFA: D & C Green 6), alizurol purple SS (CTFA: D & C Violet No. 2), carotenoid derivatives, such as lycopene, B-carotene, bixin or capsantthin, annatto and fuchsin derivatives, DC Orange 5 and quinoline yellow.

Exemplary water-soluble dyes include, for example, methylene blue and plant extracts, such as beet juice or extracts of sorghum, of Pterocarpus soyauxii, of Monascus, of Lawsonia inermis, of Mercurialis perenis, of Helianthus aanus, of Impatiens balsamina, of Curcuma longa, of Phytolacca decandra, of Solidago aureus, of Juglans regia, of Iris germanica, of Alkanna tinctoria, of Chrozophora tinctoria or of Isatis tinctoria.

Exemplary lakes which can be used in the compositions of the present invention are, for example, lakes based on cochineal carmine or based on calcium, barium, aluminium, strontium or zirconium salts of acid dyes.

Below a certain amount of coloring agent, the composition will give rise to a coat which is sufficiently transparent or translucent to retain the natural appearance of the skin, lips or superficial body growths but the composition will not make it possible to introduce a coloring visible to the naked eye.

On the other hand, for an excessively high proportion of coloring agent, the color of the make-up coat will certainly be visible but the transparency or the translucency of the coat will be insufficient to retain the natural appearance of the skin. Furthermore, cosmetic compositions comprising an excessively high proportion of coloring agent will be insufficiently bulk transparent or translucent.

Consequently, this amount can be adjusted by taking into account the test described above in the transparency/translucency section.

Exemplary cosmetic compositions according to the present invention generally comprise from 0.05% to 20% by weight and preferably from 0.1% to 15%, and more particularly from 0.5 to 10% by weight of coloring agent.

In a specific embodiment of the present invention, the colored cosmetic composition comprises at least one water-soluble or fat-soluble dye.

In another particularly advantageous embodiment of the invention, the composition comprises, as coloring agent, solely one or more dyes which are soluble in the liquid fatty phase and is devoid of insoluble coloring agents of pigment or lake type.

This is because such compositions comprising solely soluble dyes have a good coloring power in combination with excellent transparency properties due to the absence of scattering of light by insoluble particles.

Furthermore, exemplary compositions according to the invention can comprise any ingredient conventionally used in the fields concerned and more especially in the cosmetic and dermatological and/or nail-care field, with a proviso, of course, that they do not affect the translucency or transparency required according to the invention.

These ingredients can be chosen in particular from vitamins, antioxidants, thickeners, trace elements, softeners, sequestering agents, fragrances, basifying or acidifying agents, preservatives, UV screening agents, hydrophilic or lipophilic active principles, and their mixtures.

Thus, when exemplary compositions according to the invention are more particularly intended for caring for natural nails, they can in particular incorporate, as active principles, hardening agents for keratinous substances and/or active substances for treating various conditions localized on the nail, such as, for example, onichomycosis.

In exemplary embodiments, the compositions according to the invention can also comprise ingredients commonly used in the cosmetic and/or nail-care field.

Of course, a person skilled in the art will take care to choose this or these optional additional compounds and/or their amounts so that the advantageous properties of the composition according to the invention are not, or not substantially, detrimentally affected by the addition under consideration.

Compositions of the invention can be obtained, for example, according to preparation processes conventionally used in cosmetics or in dermatology.

According to an exemplary form of the invention, use will be made, for the cosmetic formulations, of preintercalated clays, preferably montmorillonites, which will be exfoliated:

(a) either in a pregel comprising at least one of the components of the final liquid fatty phase. It can be, for example, a polar or nonpolar hydrocarbonaceous oil or a volatile or nonvolatile silicone; or

(b) in the formulation, that is to say in the presence of the other solvents and/or oils and/or polymers and/or pigments and fillers.

Whether according to form (a) or (b), exfoliation of intercalated phyllosilicates can be carried out by any means capable of resulting in delamination of at least approximately 80% by weight of the phyllosilicate. In exemplary embodiments, the shear rate required for carrying out this type of exfoliation can require a shear rate of at least 10 s⁻¹, indeed even more. The upper limit of this shear rate is not critical.

Generally, shear rate can vary from approximately 10 s⁻¹ to approximately 20,000 s⁻¹ and more particularly from approximately 100 s⁻¹ to approximately 10,000 s⁻¹. In some cases, it may be advantageous to combine this shearing with heating and/or an increase in the pressure.

As regards more particularly the shearing per se, it can be provided using various conventional devices. For example, shearing can in particular be carried out using mechanical means, by thermal impact, by change in pressure or by ultrasound. The choice of the shearing method comes within the knowledge of a person skilled in the art.

Shearing obtained by mechanical methods, such as, in particular, stirrers, homogenizers or dispersers of Moritz® or Ultraturrax® type, the mixer of Banbury® type, mixers of Brabender® type and extruders, in particular of Kneader® type, are very particularly suitable for the invention.

In the specific case of mechanical shearing, in particular in an extruder, the temperature of the medium to be exfoliated, the length of the extruder, the residence time of this medium in the extruder and the type of extruder used, namely single screw/double screw, and the like, are exemplary variables capable of controlling the shear force to be applied for the exfoliation.

In various exemplary embodiments, exfoliation is regarded as satisfactory when it provides at least 80% by weight, in particular at least 85% by weight, especially at least approximately 90% by weight, indeed even approximately 95% by weight of exfoliated and intercalated phyllosilicate.

Exfoliated phyllosilicates thus formed advantageously have a thickness varying from the thickness of individual layers to the thickness of one to five associated layers.

Compositions can be provided in various forms, according to their intended uses. Cosmetic compositions can thus be provided in any dosage or form normally used for topical application. For example, compositions can be provided in anhydrous form, in the form of an oily or aqueous solution, an oily or aqueous gel, an oil-in-water or water-in-oil emulsion, a multiple emulsion, or a dispersion of oil in water by virtue of vesicles situated at the oil/water interface or a direct or inverse emulsion.

In exemplary embodiments, compositions can be provided in the form of a cast product, in dish form or in the form of a stick, in particular in the case of lipstick, care products for the lips or foundation.

In exemplary embodiments, compositions can be provided in various other forms, for example a more or less viscous liquid, a gel or a paste.

In exemplary embodiments, cosmetic compositions can constitute, inter alia, a lipstick, a lip balm, a liquid gloss, a lipstick paste, a blush, a varnish nail, a lip pencil, a solid or liquid foundation, in particular a cast foundation, a product for caring for and/or making up natural or synthetic nails, a concealer, a product for “correcting” or “embellizing” the complexion, an eyeliner, a mascara, an eyeshadow, a product for making up the body or hair, or an antisun product or coloring product for the skin.

In exemplary embodiments, cosmetic compositions are nail varnishes.

This invention is illustrated by the following examples, which are merely for the purpose of illustration.

### EXAMPLE 1 AND COMPARATIVE EXAMPLE

Preparation of a Nail Varnish

A nail varnish according to the invention is prepared by formulating phyllosilicate made of tallow derived fatty amine modified montmorillonite (Nanomer 1.34 TCN from Nanocor).

A nail varnish from the prior art is also prepared according to the same process, by exchanging the phyllosilicate weight for weight with stearyldimethylbenzylammonium chloride modified hectorite (Bentone® 27V from Elementis).

A nitrocellulosic thixotropic gel is obtained by mixing at 25° C. under appropriate stirring:

13 g of nitrocellulose (film-forming agent);

7 g of intercalated phyllosilicates or hectorite;

0.3 g of citric acid (swelling agent);

8 g of isopropylic alcohol (volatile solvent); and

butylacetate q.s. 100 g.

100 parts of the gel thus obtained is mixed at 25° C. to 450 parts with the following non-colored, non-thixotropic base composition:

8 g of plasticizer;

24 g of a mixture of nitrocellulose and co-film forming agent;

5 g of isopropylic alcohol; and

butylacetate/ethylacetate 50/50 q.s. 100 g.

A nail varnish of the following composition is thus obtained (weight in %):

film-forming agent (nitrocellulose resin) 22%

plasticizer 6.5%

isopropylic alcohol 5.3%

phyllosicilate or hectorite 1.3%

citric acid 0.05%

butylacetate 39.2%

ethylacetate 26.1%

The glossiness of this composition, is measured with a glossiness measuring device, according to the conventional manner, with the following method.

A layer having a thickness of around 300 μm is spread on a contrast card LENETA (ref. FORM 1A PENOPAC) with an automatic spreader. The layer covers at least the white bottom of the card. The coat is left to dry. The glossiness is measured at 20° and 60° on the white base with a glossiness measuring device BYK GARDNER, ref. MICRO TRI-GLOSS. A mean value for the glossiness is obtained between 0 and 100. The measured values are listed below for each of the tested compositions.

| | | Transparent varnish |
|---|---|---|
| | Transparent varnish | prepared with the |
| | prepared with Bentone ® | phyllosilicate Nanomer I.34 |
| | 27V from Elementis | TCM from Nanocor |
| Glossy 20°/60° | 53/85 | 63/86 |

### EXAMPLE 2 AND COMPARATIVE EXAMPLE

Preparation of a Nail Varnish

In a double-screw mixer, the following compounds are mixed:

20 g of hectorite or phyllosilicate as defined in Example 1;

30 g of cellulose acetobutyrate from Eastman Chemical CAB 3810.5; and

50 g of n-ethyl-o,p-toluene sulfonamide from PAN-AMERICANA (Resimpol 8).

5 parts of the above-obtained gel are mixed with 95 parts of the nitrocellulosic thixotropic gel described in Example 1.

A layer having a thickness of around 300 μm is spread on a contrast card LENETA (ref. FORM 1A PENOPAC) with an automatic spreader. The layer covers at least the white bottom of the card. The coat is left to dry. The glossiness is measured at 20° and 60° on the white base with a glossiness measuring device BYK GARDNER, ref. MICRO TRI-GLOSS. A mean value for the glossiness is obtained between 0 and 100. The measured values are listed below for each of the tested compositions.

The obtained results for each of the tested compositions are listed below.

| | | Transparent varnish |
|---|---|---|
| | Transparent varnish prepared | prepared with the |
| | with Bentone ® 27V from | phyllosilicates Nanomer |
| | Elementis | from Nanocor I34.TCN |
| Glossy 20°/60° | 54/79 | 71/87 |

While this invention has been described in conjunction with the exemplary embodiments and examples outlined above, various alternatives, modifications, variations, improvements and/or substantial equivalents, whether known or that are or may be presently unforeseen, may become apparent to those having at least ordinary skill in the art. Accordingly, the exemplary embodiments of the invention, as set forth above, are intended to be illustrative, not limiting. Various changes may be made without departing from the spirit and scope of the invention. Therefore, the invention is intended to embrace all known or later developed alternatives, modifications, variations, improvements and/or substantial equivalents.

What is claimed is:

1. A cosmetic composition for caring for and/or making up skin, lips and/or superficial body growths, comprising:
at least one phase chosen from a liquid fatty phase and a water-immiscible organic phase;
wherein the phase is structured by at least an effective amount of exfoliated phyllosilicates derived from at least one intercalated phyllosilicate.

2. The composition according to claim 1, wherein the exfoliated phyllosilicates have an aspect ratio varying from 50 to 2000.

3. The composition according to claim 2, wherein the exfoliated phyllosilicates have an aspect ratio varying from 75 to 1500.

4. The composition according to claim 3, wherein the exfoliated phyllosilicates have an aspect ratio varying from 200 to 1000.

5. The composition according to claim 1, wherein at least 50% of the exfoliated phyllosilicates are formed of less than ten sheets of phyllosilicate.

6. The composition according to claim 5, wherein at least 70% of the exfoliated phyllosilicates are formed of less than ten sheets of phyllosilicate.

7. The composition according to claim 6, wherein at least 90% of the exfoliated phyllosilicates are formed of less than ten sheets of phyllosilicate.

8. The composition according to claim 7, wherein at least 95% of the exfoliated phyllosilicates are formed of less than ten sheets of phyllosilicate.

9. The composition according to claim 1, wherein at least 50% of the exfoliated phyllosilicates are formed of less than 5 sheets of phyllosilicate.

10. The composition according to claim 9, wherein at least 70% of the exfoliated phyllosilicates are formed of less than 5 sheets of phyllosilicate.

11. The composition according to claim 10, wherein at least 90% of the exfoliated phyllosilicates are formed of less than 5 sheets of phyllosilicate.

12. The composition according to claim 11, wherein at least 95% of the exfoliated phyllosilicates are formed of less than 5 sheets of phyllosilicate.

13. The composition according to claim 1, wherein at least 50% of the exfoliated phyllosilicates are formed of less than 3 sheets of phyllosilicate.

14. The composition according to claim 13, wherein at least 70% of the exfoliated phyllosilicates are formed of less than 3 sheets of phyllosilicate.

15. The composition according to claim 14, wherein at least 90% of the exfoliated phyllosilicates are formed of less than 3 sheet of phyllosilicate.

16. The composition according to claim 15, wherein at least 95% of the exfoliated phyllosilicates are formed of less than 3 sheets of phyllosilicate.

17. The composition according to claim 1, wherein at least 50% of the exfoliated phyllosilicates are formed of a single sheet of phyllosilicate.

18. The composition according to claim 17, wherein at least 70% of the exfoliated phyllosilicates are formed of a single sheet of phyllosilicate.

19. The composition according to claim 18, wherein at least 90% of the exfoliated phyllosilicates are formed of a single sheet of phyllosilicate.

20. The composition according to claim 19, wherein at least 95% of the exfoliated phyllosilicates are formed of a single sheet of phyllosilicate.

21. The composition according to claim 1, wherein the exfoliated phyllosilicates are present in an amount of from 0.05 to 30% by weight with respect to the total weight of the composition.

22. The composition according to claim 21, wherein the exfoliated phyllosilicates are present in an amount of from 0.1 to 20% by weight with respect to the total weight of the composition.

23. The composition according to claim 22, wherein the exfoliated phyllosilicates are present in an amount of from 0.5 to 10% by weight with respect to the total weight of the composition.

24. The composition according to claim 1, wherein the exfoliated phyllosilicates are derived from intercalation of smectites.

25. The composition according to claim 24, wherein the smectites are chosen from the group consisting of montmorillonites, nontronites, beidellites, volkonskoites, hectorites, saponites, sauconites, sobockites, stevensites, svinfordites and vermiculites.

26. The composition according to claim 25, wherein the smectites comprise two or more members chosen from the group consisting of montmorillonites, nontronites, beidellites, volkonskoites, hectorites, saponites, sauconites, sobockites, stevensites, svinfordites and vermiculites.

27. The composition according to claim 1, wherein the exfoliated phyllosilicates comprise at least 15% by weight of intercalating agent with respect to the weight of dry phyllosilicate comprising less than 5% of water.

28. The composition according to claim 27, wherein the exfoliated phyllosilicates comprise at least 20% by weight of intercalating agent with respect to the weight of dry phyllosilicate comprising less than 5% of water.

29. The composition according to claim 28, wherein the exfoliated phyllosilicates comprise at least 30% by weight of intercalating agent with respect to the weight of dry phyllosilicate comprising less than 5% of water.

30. The composition according to claim 1, wherein the exfoliated phyllosilicates comprise an intercalating agent chosen from the group consisting of oniums having at least one C₁ to C₅₀ hydrocarbonaceous chain, polymeric organic compounds and nonpolymeric organic compounds.

31. The composition according to claim 30, wherein the polymeric organic compound has at least one group chosen the group consisting from aromatic rings and polar groups.

32. The composition according to claim 30, wherein the nonpolymeric organic compound has at least one group chosen from the group consisting of aromatic rings and polar groups.

33. The composition according to claim 31, wherein the group is chosen from the group consisting of carboxyl, hydroxyl, polyol, carboxylic acid, aldehyde, ketone, linear amine, cyclic amine, linear amide, cyclic amide, ester, lactone and ether groups.

34. The composition according to claim 32, wherein the group is chosen from the group consisting of carboxyl, hydroxyl, polyol, carboxylic acid, aldehyde, ketone, linear or cyclic amine, linear or cyclic amide, ester, lactone and ether groups.

35. The composition according to claim 30, wherein the onium cation is chosen from the group consisting of primary, secondary, tertiary or quaternary ammoniums having at least one C₄-C₅₀ alkyl chain.

36. The composition according to claim 30, wherein the nonpolymeric organic compound has a polar group and at least one hydrophobic chain.

37. The composition according to claim 36, wherein the hydrophobic chain is chosen from the group consisting of C₄ to C₅₀ alkyl, C₄ to C₅₀ alkylene and C₄ to C₅₀ alkylaryl chains.

38. The composition according to claim 30, wherein the nonpolymeric organic compound is chosen from the group consisting of alkylpyrrolidones with a C₄ to C₅₀ alkyl chain.

39. The composition according to claim 38, wherein the nonpolymeric organic compound is chosen from the group consisting of alkylpyrrolidones with C₈ to C₃₀ alkyl chain.

40. The composition according to claim 30, wherein the polymeric organic compound is a synthetic oligomer chosen from the group consisting of polyvinylpyrrolidone (PVP) derivatives, polyvinyl alcohol (PVA) derivatives, polyalkylenoxide derivatives, polyacrylic derivatives in their polymeric and copolymeric form, polymethacrylic acid (PMAA) derivatives, polyvinyloxazolidone (PVO) and polyvinylmethyloxazolidone (PVMO) derivatives and polyvinyloxazoline derivatives.

41. The composition according to claim 30, wherein the polymeric organic compound is a synthetic polymer chosen from the group consisting of polyvinylpyrrolidone (PVP) derivatives, polyvinyl alcohol (PVA) derivatives, polyalkylenoxide derivatives, polyacrylic derivatives in their polymeric and copolymeric form, polymethacrylic acid (PMAA) derivatives, polyvinyloxazolidone (PVO) and polyvinylmethyloxazolidone (PVMO) derivatives and polyvinyloxazoline derivatives.

42. The composition according to claim 1, wherein the composition is in a form of emulsion.

43. The composition according to claim 42, wherein the emulsion is a direct or inverse emulsion.

44. The composition according to claim 42, wherein the emulsion is in a form chosen from the group consisting of water-in-oil, oil-in-water, and multiple emulsions.

45. The composition according to claim 42, wherein the emulsion comprises at least one emulsifying agent.

46. The composition according to claim 45, wherein the emulsion comprises at least one co-emulsifying agent.

47. The composition according to claim 46, wherein the emulsifying and co-emulsifying agents are present in an amount of less than 30% by weight relative to the total weight of the composition.

48. The composition according to claim 47, wherein the emulsifying and co-emulsifying agents are present in an amount of less than 20% by weight relative to the total weight of the composition.

49. The composition according to claim 48, wherein the emulsifying and co-emulsifying agents are present in an amount of less than 15% by weight relative to the total weight of the composition.

50. The composition according to claim 49, wherein the emulsifying and co-emulsifying agents are present in an amount of less than 0.5% by weight relative to the total weight of the composition.

51. The composition according to claim 42, wherein the emulsion is free of any emulsifying and co-emulsifying agents.

52. The composition according to claim 1, comprising a continuous fatty phase.

53. The composition according to claim 1, wherein the composition is anhydrous.

54. The composition according to claim 1, wherein the fatty phase comprises at least one fatty substance which is liquid at ambient temperature and at atmospheric pressure.

55. The composition according to claim 54, wherein the fatty phase comprises at least one fatty substance which is solid at ambient temperature and at atmospheric pressure.

56. The composition according to claim 54, wherein the fatty substance which is liquid at ambient temperature and at atmospheric pressure comprises at least one oil chosen from volatile oils and nonvolatile oils.

57. The composition according to claim 56, wherein the oil is a mixture of at least one volatile oil and at least one nonvolatile oil.

58. The composition according to claim 56, wherein the nonvolatile oil is chosen from the group consisting of hydrocarbonaceous oils of animal origin; vegetable hydrocarbonaceous oils; linear or branched hydrocarbons of mineral or synthetic origin; synthetic ethers having from 10 to 40 carbon atoms; synthetic esters, polyol esters; fatty alcohols which are liquid at ambient temperature having from 12 to 26 carbon atoms; higher fatty acids; and silicone oils of polymethylsiloxane (PDMS) type.

59. The composition according to claim 58, wherein the nonvolatile oil is a mixture of at least two members chosen from the group consisting of hydrocarbonaceous oils of animal origin; vegetable hydrocarbonaceous oils; linear or branched hydrocarbons of mineral or synthetic origin; synthetic ethers having from 10 to 40 carbon atoms; synthetic esters, polyol esters; fatty alcohols which are liquid at ambient temperature having from 12 to 26 carbon atoms; higher fatty acids; and silicone oils of polymethylsiloxane (PDMS) type.

60. The composition according to claim 56, wherein the volatile oil is chosen from the group consisting of hydrocarbonaceous oils having from 8 to 16 carbon atoms and volatile silicone oils.

61. The composition according to claim 60, wherein the volatile oil is a mixture of at least one hydrocarbonaceous oil having from 8 to 16 carbon atoms and at least one volatile silicone oil.

62. The composition according to claim 56, wherein the composition comprises an oily phase present in an amount of from 1 to 80% by weight relative to the total weight of the composition.

63. The composition according to claim 62, wherein the oily phase is present in an amount of from 1 to 50% by weight relative to the total weight of the composition.

64. The composition according to claim 1, wherein the water immiscible organic phase comprises at least one organic solvent having a miscibility in water of less than or equal to 50% by weight.

65. The composition according to claim 64, wherein the solvent is chosen from the group consisting of short-chain esters having from 4 to 8 carbon atoms in total.

66. The composition according to claim 65, wherein the solvent is chosen from the group consisting of ethyl acetate, propyl, butyl and aryl acetates, and isopentyl acetate.

67. The composition according to claim 66, wherein the solvent is a mixture of at least two members chosen from the group consisting of the ethyl acetate, the propyl, the butyl and the aryl acetates, and the isopentyl acetate.

68. The composition according to claim 1, comprising at least one coloring agent.

69. The composition according to claim 68, wherein the coloring agent is chosen from the group consisting of water-soluble dyes, fat-soluble dyes, pigments and lakes.

70. The composition according to claim 69, comprising from 0.05 to 20% by weight of the coloring agent.

71. The composition according to claim 70, comprising from 0.1 to 15% by weight of coloring agent.

72. The composition according to claim 71, comprising from 0.5 to 10% by weight of coloring agent.

73. The composition according to claim 1, wherein the composition is transparent, translucent or capable of giving a transparent or translucent coat.

74. The composition according to claim 1, wherein the composition is in the form of a product cast.

75. The composition according to claim 62, wherein the product cast is chosen from the group consisting of sticks and dishes.

76. The composition according to claim 1, wherein the composition is provided in a form chosen from the group consisting of lipsticks, lip balms, cast foundations, products for caring for and/or making up synthetic or natural nails, concealers, products for “correcting” or “embellishing” the complexion, eyeshadows and blushes.

77. The composition according to claim 1, wherein the composition is a nail varnish.

78. A method for texturizing a liquid fatty phase of a cosmetic composition for caring for and/or making up skin, lips and/or superficial body growths comprising adding exfoliated phyllosilicates derived from at least one intercalated phyllosilicate to the liquid fatty phase.

79. The method according to claim 78, wherein the composition is an emulsion.

80. The method according to claim 79, wherein the composition is transparent, translucent or capable of giving a transparent or translucent coat.

81. A method for stabilizing a cosmetic emulsion comprising preparing the cosmetic emulsion with exfoliated phyllosilicates derived from at least one intercalated phyllosilicate.

82. A process for making up skin, lips and/or superficial body growths comprising applying at least one layer of the composition according to claim 1 to the skin, lips and/or superficial body growths.

83. A made-up synthetic substrate comprising the composition according to claim 1 on all or part of its surface.
